(19) United States
(12) Patent Application Publication
(10) Pub. No.: US 2025/0073440 A1
(43) Pub. Date: Mar. 6, 2025

(54) TRANSPORT ELEMENT FOR TRANSPORTING A SUBSTANCE CONTAINED IN A RESERVOIR, INFUSION SET AND GLUCOSE REGULATING SYSTEM COMPRISING THE FORMER AND METHOD OF CONNECTING SUCH A TRANSPORT ELEMENT TO A RESERVOIR

(71) Applicant: INREDA DIABETIC B.V., Goor (NL)

(72) Inventors: Robin KOOPS, Goor (NL); Mickael Gilbert Robert BOULAY, Goor (NL); Roy Hermannus Johannes Zwijnenberg, Goor (NL)

(73) Assignee: INREDA DIABETIC B.V., Goor (NL)

(21) Appl. No.: 18/580,451

(22) PCT Filed: Jul. 19, 2022
(86) PCT No.: PCT/EP2022/070128

§ 371 Date: Jan. 18, 2024

(30) Foreign Application Priority Data
Jul. 22, 2021 (NL) 2028804

(51) Int. Cl. A61M 39/10

(52) U.S. Cl. CPC A61M 39/1011

(57) ABSTRACT

A transport element for transporting a substance in a reservoir includes a transport means, such as a conduit, tube, hose or the like, and a connector arranged at an inlet end of the transport means for connecting to the reservoir such that in a connected condition in which the connector is connected to said reservoir a medium through flow connection is provided between said reservoir and the inlet end of the transport means for receiving said substance from said reservoir. The connector includes aligning and holding means for aligning and holding the reservoir in a predetermined orientation with respect to and/or position along a longitudinal axis of the connector. An infusion set, system or combination including transport element, and a method of at least fluidly connecting such a transport element to a reservoir.

The invention relates to a transport element for transporting a substance contained in a reservoir, said transport element being arranged to be part of an infusion set, said transport element comprising a transport means, such as a conduit, tube, hose or the like, and a connector arranged at an inlet end of the transport means for connecting to said reservoir such that in a connected condition in which the connector is connected to said reservoir a medium through flow connection is provided between said reservoir and the inlet end of the transport means for receiving said substance from said reservoir.

Such a transport element is known per se and may be used by diabetes patients as a part of a system for regulating the concentration of glucose in the blood of a person, e.g. with a substance such as or comprising insulin or glucagon. An example of such a system can be found in the international patent application WO 2007/049961, but the transport element may also be used in any other type of a system for regulating the concentration of glucose in the blood of a person.

With currently known transport elements, it requires specific attention to reliably connect the reservoir, which is typically embodied as an ampule, to the connector of the transport element. This is of particular importance, since the process of connecting is a task which is typically carried out by patients themselves, not by trained medical professionals. When the reservoir is connected in an incorrect manner, malfunctions such as leakages may occur.

It is an object of this invention to at least partially overcome these disadvantages and/or to improve said known transport element.

This object is met by a transport element according to the preamble, wherein said connector comprises aligning and holding means for aligning and holding the reservoir in a predetermined orientation with respect to and/or position along a longitudinal axis of the connector.

The provision of such aligning and holding means, i.e. means which are arranged for both aligning and holding the reservoir in a predetermined orientation with respect to and/or position along such a longitudinal axis, reduces the chance of misalignment and/or inadequate positioning of the reservoir with respect to the connector. Said longitudinal axis may correspond to the intended direction of insertion of said reservoir into the connector or may alternatively, where applicable, be substantially parallel with respect to and/or may coincide with the longitudinal axis of a penetrating means, as will be discussed below. Said predetermined orientation may be substantially parallel to said longitudinal axis, i.e. a longitudinal axis of said reservoir may be substantially parallel to the longitudinal axis of said connector. Said position may in particular be a position along the longitudinal axis in which the reservoir has a substantially leak tight connection with the connector. An example of such a position will be discussed with respect to an exemplary embodiment discussed in the following.

In said predetermined orientation and/or position said reservoir may be connected to the connector in such a manner that it is rotatable with respect to the connector, preferably about the longitudinal axis of the connector, while being held by the aligning and holding means such that it can only be removed from the connector by overcoming the holding force by which the reservoir is held by the aligning and holding means.

The aligning and holding means can be made into a wide variety of embodiments, for example for connecting to different types of reservoirs, typically embodied as an ampule. In particular, the connector of the transport element according to the current invention may connect to a reservoir with a standard featureless end. A featureless end may be defined here as an end which does not comprise features intended or primarily intended for connecting the reservoir to the connector, such as notches on a head portion of the reservoir specifically designed to interact with corresponding parts in specific types of connectors. As a consequence, the versatility of the connector of the transport element is increased. As an example, the ampule may comprise a neck portion wherein the aligning and holding means are arranged to protrude into an area of and/or engage said neck portion.

The connector of the transport element according to the invention is typically made of a plastic.

The connector and the transport means may either be one integral part or may be separate elements that may be connectable to each other to form the transport element.

In a preferred embodiment of the transport element according to the invention, the connector of the transport element comprises additional aligning means, said additional aligning means extending before and/or after the aligning and holding means as seen in the direction of the longitudinal axis.

Before and after can be seen in said insertion direction of the reservoir into the connector, which insertion direction is parallel to the longitudinal direction. In other words, the additional aligning means may extend upstream and/or downstream of the aligning and holding means as seen in said insertion direction. In other words, the additional aligning means may extend beyond the aligning and holding means in one or both directions as seen in the longitudinal direction.

The additional aligning means may for example be continuously formed, such that these extend from before the aligning and holding means, along the aligning and holding means and after the aligning and holding means. Alternatively the additional aligning means may comprise one, two, or even more separate parts, wherein if one part is provided that one part may extend either before or after the aligning and holding means and if two parts are provided one part thereof may extend before and the other part may extend after the aligning and holding means.

Said additional aligning means extending before the aligning and holding means may in particular be arranged for pre-aligning the reservoir with respect to the longitudinal axis of the connector in a first part of a connection path of connecting the reservoir to the connector, wherein in a second, subsequent part of said connection path said reservoir is aligned by said aligning and holding means and optionally said additional aligning means, and is subsequently held by the aligning and holding means at the end of the connection path in the connected condition.

An advantage thereof is that the reservoir is already pre-aligned with respect to the longitudinal axis when it contacts the aligning and holding means, in order to bring the reservoir into an orientation which is at least relatively close and preferably identical to the predetermined orientation prior to being aligned and held by the aligning and holding means. It is noted in this respect that if the reservoir is pre-aligned in the predetermined orientation it can alternatively be said that the aligning and holding means are arranged to maintain the alignment of the reservoir in said predetermined orientation.

Said additional aligning means extending after the aligning and holding means may be arranged for maintaining the alignment of said reservoir in said predetermined orientation in the connected condition.

This may alternatively be formulated as said additional aligning means extending after the aligning and holding means being arranged for post-aligning the reservoir in the predetermined orientation.

In a preferred embodiment of the transport element according to the invention, the aligning and holding means are more flexible than the additional aligning means.

An advantage of such flexibility of the aligning and holding means is that the reservoir may be easily connected thereto. An advantage of the additional aligning means being less flexible is that this may enhance the alignment of the reservoir in the predetermined orientation.

More flexible may for example mean that the aligning and holding means are able to, preferably resiliently, move or displace when the reservoir is connected to the transport element, wherein the additional aligning means preferably do not move or displace when the reservoir is connected to the transport element.

In particular the additional aligning means may be embodied as rigid and/or stiff and/or non-flexible additional aligning means. This may be achieved by means of a suitable material choice and/or design choice, such as a suitable shape and/or thickness, of the additional aligning means.

In particular the aligning and holding means may be embodied as flexible and/or resilient aligning and holding means. This may be achieved by means of a suitable material choice and/or design choice, such as a suitable shape and/or thickness, of the aligning and holding means.

An advantage of achieving the difference in flexibility as a result of a different design of the aligning and holding means in comparison to the additional aligning means is that these may be made of the same material, which may preferably the same material from which the whole connector is made.

In a preferred embodiment of the transport element according to the invention, the additional aligning means comprise at least one elongated rib that protrudes inwardly from the inner side of a circumferential wall of said connector and extends substantially parallel to said longitudinal axis.

Such a rib, which is configured to touch a part of the reservoir with a protruding edge thereof in order to obtain pre-alignment and/or post-alignment of the reservoir, is relatively simple in construction, and is thereby easy to produce. It may for instance be made integrally with the circumferential wall of the connector, e.g. by injection moulding. This may especially be the case if the rib and the circumferential wall are made of the same material, typically a plastic. If the additional aligning means extend on both sides of the aligning and holding means as seen along the longitudinal axis, said rib is preferably continuously formed, which may mean that it is at least not interrupted by said aligning and holding means.

Said at least one rib may in particular protrude in a radial direction from the inner side of the circumferential wall of the connector.

In particular the at least one rib may extend substantially parallel to the longitudinal axis with a longitudinal axis thereof.

The circumferential wall of the connector may have a substantially cylindrical shape, at least the inner side thereof. Such a shape may conveniently accommodate a cylindrical neck and/or head portion of a reservoir.

In a preferred embodiment of the transport element according to the invention, the additional aligning means comprise a plurality of said ribs distributed along the circumference of the circumferential wall, preferably in a uniform distribution.

By providing a plurality of ribs that are distributed along the circumference of the circumferential wall, preferably in said uniform distribution, the reservoir can be centred within the connector. In such an embodiment the reservoir is arranged coaxially with the connector in the predetermined orientation, i.e. the longitudinal axis of the reservoir coincides with the longitudinal axis of the connector. The number of ribs may for instance be at least three, at least four, at least five, at least six, or even more. The number of ribs is typically determined by a person skilled in the art based on the requirements for the connector, for example dependent on the size thereof.

The angular distance between neighbouring ribs is preferably the same for the various ribs and may be calculated by dividing 360 degrees by the number of ribs.

In a preferred embodiment of the transport element according to the invention, the aligning and holding means comprise at least one resilient member, said resilient member having a first state for holding said reservoir and being moveable to a second state when said reservoir is pushed against said resilient member when said reservoir is connected to or disconnected from said connector, and which resilient member is elastically urged to its first state.

An advantage of such a resilient member that is elastically urged to its first state and that is temporarily moveable to its second state is during connecting the reservoir to and disconnecting the reservoir from the connector is that the reservoir may easily be connected thereto and disconnected therefrom by temporarily moving the resilient member to the second state, while said reservoir may be accurately held in said predetermined orientation and/or position in the connected condition in which the resilient member is in its first state.

In a preferred embodiment of the transport element according to the invention, said at least one resilient member comprises at least one substantially crescent-shaped member, wherein the substantially crescent-shaped member is attached to the inner side of a or the circumferential wall of the connector with two opposing end zones thereof and wherein a convex side of said substantially crescent-shaped member that extends between the two opposing end zones protrudes inwardly from the inner side of said circumferential wall of the connector.

Such a substantially crescent-shaped resilient member is an advantageous embodiment of a resilient member, since it has been found that such a crescent-shaped member is a particular example of a resilient member which is well able to reliably hold the reservoir, in particular if multiple substantially crescent-shaped members are provided. An alternative or additional advantage of such a crescent-shaped member is that it may substantially maintain its shape in the first state, even after repeatedly connecting and disconnecting a reservoir to and from the connector.

A substantially crescent-shaped member may alternatively be defined as an arcuate member, or a member comprising a mid-zone between said end zones, which mid-zone is closer to the reservoir than said end zones, i.e. extend radially more inwards than said end zones, preferably both in a non-connected condition, as well as in the connected condition. Substantially crescent-shaped may in particular mean that such a mid-zone is at least partially, but preferably entirely, curved.

During connecting to or disconnecting the reservoir from the connector the convex side of the crescent-shaped member may be pressed by the reservoir into the direction of the circumferential wall, which is the before called second state of the resilient member, and may return to its crescent or arcuate shape when the reservoir is connected to or removed from the connector.

The shape of the substantially crescent-shaped member is in particular substantially crescent-shaped in a plane substantially orthogonal to the longitudinal axis of the connector.

The end zones of the substantially crescent-shaped member are preferably fixed to the circumferential wall at substantially the same position along the longitudinal axis of said connector, i.e. the substantially crescent-shaped member extends substantially orthogonal to the longitudinal axis. In other words, said substantially crescent-shaped member is bound by a plane substantially perpendicular to the longitudinal axis. Such a configuration increases the grip of said crescent-shaped member on a reservoir, in particular when the reservoir comprises a narrowed portion in the end of the reservoir, such as a neck portion, and when the connector is designed to hold the reservoir in the connected condition by said narrowed portion.

In a preferred embodiment of the transport element according to the invention, a hole or opening is provided in said circumferential wall in an area between said two opposing end zones of the at least one substantially crescent-shaped member.

This may increase the ease of production of such a connector, especially when the connector is produced using injection moulding.

If multiple substantially crescent-shaped members are provided a such hole or opening may be provided between the opposing end zones of any one or more of the substantially crescent-shaped members.

In a preferred embodiment of the transport element according to the invention, the transport element comprises a plurality of said resilient members and wherein the resilient members are distributed along the circumference of a or the circumferential wall, preferably in a uniform distribution and/or arranged at a substantially same position along the longitudinal axis.

Providing a plurality of said resilient members improves the alignment and holding of the reservoir. The resilient members may together define a ring of resilient members.

Preferably, the connector comprises a plurality of said substantially crescent-shaped members. The end zones of the substantially crescent-shaped member are preferably fixed to the circumferential wall at substantially the same position along the longitudinal axis of said connector.

When the additional aligning means comprise a plurality of ribs, the number of ribs may be equal to the number of resilient members of the connector.

The ribs and resilient members are preferably provided as separate, individual elements.

In a preferred embodiment of the transport element according to the invention, the ribs and resilient members are arranged alternatingly along the circumference of the circumferential wall, preferably in an uniform distribution and/or spaced apart from each other.

Providing the ribs and resilient members in an alternating manner and preferably in an even distribution along the circumference of the circumferential wall may aid the correct alignment of the reservoir.

In a preferred embodiment of the transport element according to the invention, said connector comprises a penetrating means, such as a needle, that extends substantially parallel to the longitudinal axis with a free end thereof in the direction of the aligning and holding means for penetrating a membrane or the like of said reservoir in at least the connected condition, wherein said aligning and holding means and optionally said additional aligning means are configured for aligning the reservoir in a predetermined orientation with respect to said penetrating means such that the penetrating means are oriented substantially perpendicular to the membrane of said reservoir during penetrating the membrane during connecting of the connector to said reservoir, and for holding said reservoir in said predetermined orientation and in such a position that the penetrating means substantially completely penetrates the membrane in the connected condition.

A typically used reservoir comprises a piercable membrane for closing said reservoir. In accordance with the invention said membrane is pierced in a substantially orthogonal manner by the penetrating means, thereby reducing or even preventing leakage of substance.

Orthogonal may be defined between the local plane or area of the membrane where it is penetrated and the longitudinal direction of the penetrating means.

Said needle is in particular a hollow needle for transporting said substance there through.

The position of the reservoir in which the reservoir is held with respect to the connector, in this respect also referred to as the depth of the reservoir, is chosen such that the penetrating means, in the connected condition, fully penetrate the membrane of the reservoir in order to reduce or prevent leakage of substance and to provide a correct fluid connection between the transport element and the reservoir. Such a position or depth of the reservoir with respect to the connector may be suitably chosen by arranging the aligning and holding means at such a position along the longitudinal axis with respect to the penetrating means that the penetrating means will completely penetrate the membrane in the connected condition. For example, the penetrating end or free end of the penetrating means may be located in the vicinity and for example slightly above the aligning and holding means as seen in the direction of insertion of the reservoir, such that in the connected condition in which a head portion of the reservoir extends above the aligning and holding means as seen in the direction of insertion the membrane that is comprised by the head portion is completely penetrated.

The aligning and holding means and, where applicable, the additional aligning means, are preferably configured for centring the reservoir with respect to the penetrating means during connecting the reservoir to the connector and/or in the connected condition.

In a preferred embodiment of the transport element according to the invention, the aligning and holding means are configured to protrude into an area of and/or grip a neck portion of said reservoir in the connected condition.

When the aligning and holding means are configured to protrude into an area of and/or grip such a neck portion, which is narrowed with respect to a broader end or head portion of the reservoir, the reservoir is held by the aligning and holding means in a reliable manner.

The invention further relates to an infusion set, comprising:
a transport element as described above in any of the above described embodiments and/or comprising any feature as described above, alone or in any suitable combination, further comprising an additional connector arranged at an outlet end of the transport means, and
an infusion element comprising an infusion means for infusing a substance into a body of a person, said infusion means for example being a cannula, catheter, needle, or nozzle, said infusion means comprising an inlet end for receiving said substance and an outlet end for infusing said substance into said body, wherein said infusion element comprises a connector for connecting to said additional connector of said transport element of the infusion set, wherein said additional connector of said transport element is connectable to said connector of said infusion element such that in a connected condition in which the additional connector of the transport element is connected to the connector of the infusion element a medium through flow connection is provided between the outlet end of the transport means and the inlet end of the infusion means for feeding the substance to the infusion means.

The transport element according to the present invention may thus be used as a part of an infusion set, allowing the substance contained in the reservoir to be infused into said body via said infusion set.

In order to distinguish between the different connectors of said infusion set, the connector of the infusion element may be referred to as the first connector, whereas the additional connector of the transport element that is connectable to said first connector may be referred to as the second connector. The connector connectable to the reservoir is than referred to as the third connector.

The invention further relates to a system for regulating the concentration of glucose in the blood of a person, comprising at least one infusion set as described above and a device for selectively supplying at least one substance contained in at least one reservoir, said device comprising:
at least one receiving space, for accommodating a said reservoir;
at least one port, for connecting to a said connector of a said transport element of the infusion set, and
a controller for controlling said device such that a certain amount of a said substance is supplied and thereby infused into the body of the person.

The at least one port is arranged in vicinity of the at least one receiving space, for example at or near an insert opening of the receiving space via which the reservoir may be inserted.

In a connected condition in which the connector of the transport element is connected to the port of the device, said connector is also connected to the reservoir contained in the receiving space.

The connection to the port provides a mechanical connection between the device and the infusion set, while the connection to the reservoir provides said fluid through flow connection between the reservoir and the infusion set.

The device is not restricted to just one receiving space and may thus comprise more than one receiving space. In the same way, the device may comprise more than one port, preferably one port per receiving space. Such a device may be used with one or more than one infusion set, preferably one infusion set for each receiving space. In particular, the number of receiving spaces and ports may be two, wherein a first receiving space may serve to accommodate a reservoir containing insulin, whereas a second receiving space may serve to accommodate a reservoir containing glucagon.

The connector of the transport element according to the invention typically also comprises means for connecting the connector to said port of the device. For example said connector bayonet may be of the bayonet type.

In a preferred embodiment of the system according to the invention, the system is configured for inserting the reservoir in said receiving space of the device after connecting the outlet end of the reservoir to the connector of the transport element, thereby aligning and holding said reservoir with the aligning and holding means of the connector prior to inserting the reservoir into the receiving space.

The invention further relates to a combination of a reservoir, preferably a reservoir pre-filled with a substance such as or comprising insulin or glucagon, and:
a transport element as described above in any of the above described embodiments and/or comprising any feature as described above, alone or in any suitable combination;
an infusion set as described above, or
a system as described above.

The reservoir is preferably a reservoir which is pre-filled with a said substance. In order to regulate the concentration of glucose in the blood of a person, said substance preferably is or comprises insulin or glucagon.

The invention further relates to a method of at least fluidly connecting a transport element to a reservoir, comprising the steps of:
a) providing a transport element as described above in any of the above described embodiments and/or comprising any feature as described above, alone or in any suitable combination, an infusion set as described above or a system as described above;
b) providing a reservoir comprising an outlet end;
c) connecting the outlet end of the reservoir to the connector of the transport element, thereby aligning and holding said reservoir with the aligning and holding means of the connector in a connected condition.

Preferably, in step c) said reservoir is connected to the connector along a connection path, wherein in a first part of said path the reservoir is pre-aligned by said additional aligning means, wherein in a second, subsequent part of said path the reservoir is aligned by said aligning and holding means and optionally the additional aligning means, and wherein said reservoir is subsequently held by said aligning and holding means in said connected condition and optionally aligned by said additional aligning means.

In a preferred embodiment of the method according to the invention, in step a) a system as described above is provided, and the method further comprises step d) of inserting the reservoir in said receiving space of the device for selectively supplying at least one substance, wherein step d) is preferably executed after step c).

Executing step d) after step c) allows the reservoir to be aligned and held by the connector of the transport prior to being inserted into the receiving space.

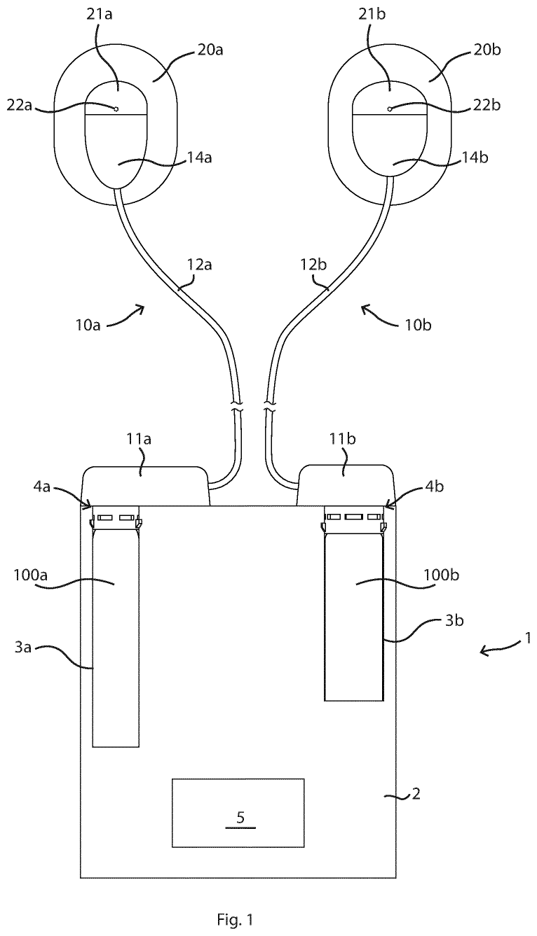

The invention will be further explained with respect to FIGS. 1 to 6, wherein:

FIG. 1 provides a schematic overview of a system according to a first embodiment of the invention.

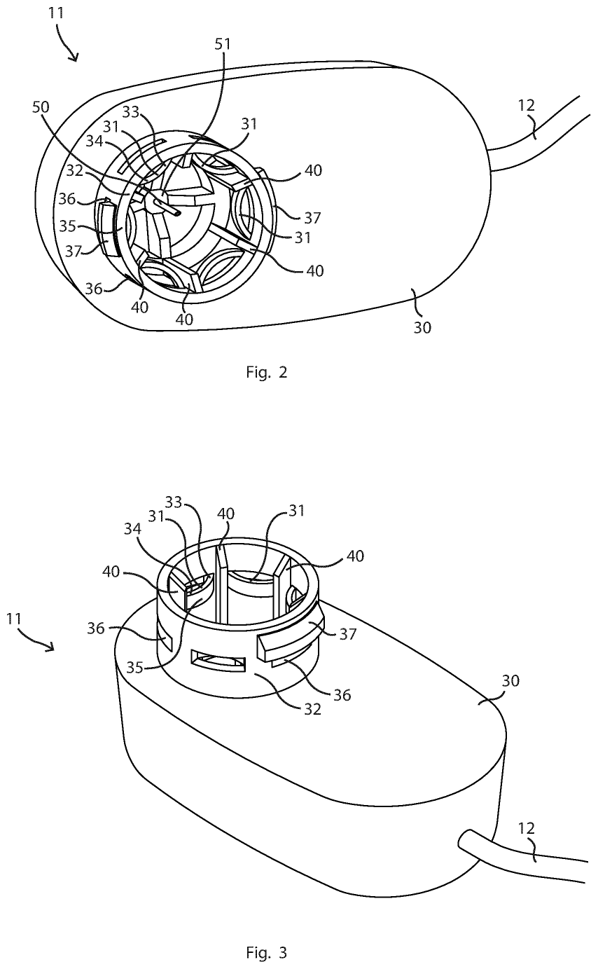

FIG. 2 shows a perspective view of a connector comprise by a transport element of the system according to FIG. 1.

FIG. 3 shows another perspective view of the connector according to FIG. 2.

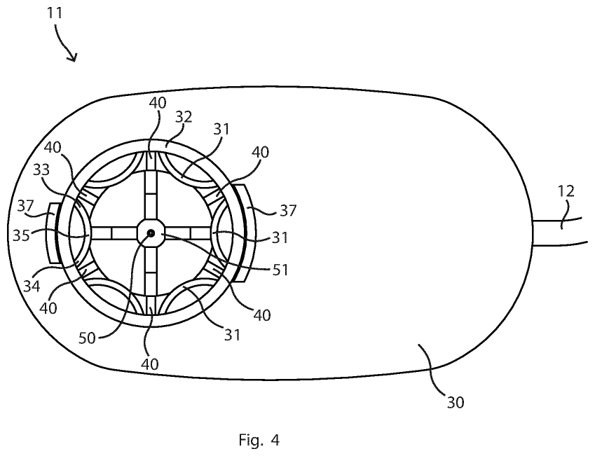

FIG. 4 shows a bottom view of the connector according to FIG. 2.

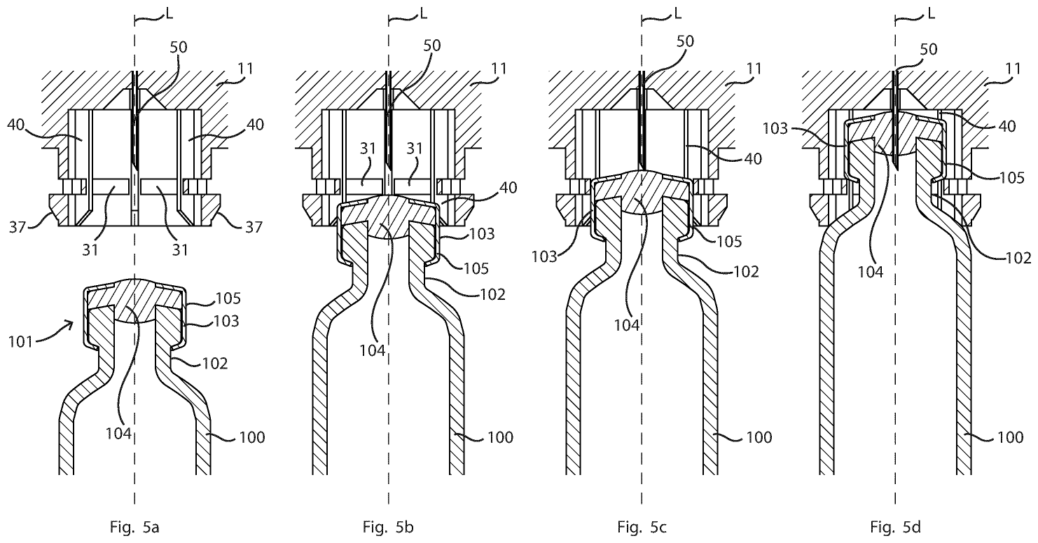

FIGS. 5A, 5B, 5C and 5D show the connector according to FIG. 2 during the process of inserting the reservoir into said connector.

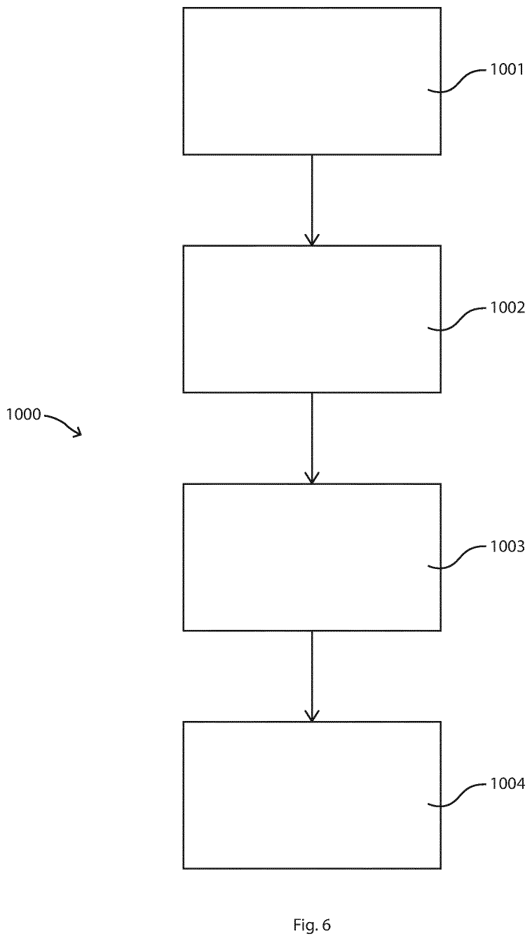

FIG. 6 shows a method according to the invention.

In FIG. 1, a system 1 for regulating the concentration of glucose in the blood of a person is shown. The system 1 comprises a device 2 with a first receiving space 3a for accommodating a first reservoir 100a and a second receiving space 3b for accommodating a, second reservoir 100b. The reservoirs 100a,100b may be embodied as a so-called ampule and may contain a respective substance, in particular a substance affecting the glucose concentration in the blood of a person. For example, the reservoir 100a may contain insulin and the reservoir 100b may contain glucagon. The device 2 comprises two ports 4a, 4b for connecting a respective transport element 10a, 10b, in particular a respective connector 11a, 11b thereof, thereto. Said connectors 11a, 11b are arranged at an inlet end of a respective transport means 12a, 12b of a respective transport element 10a, 10b. In this example of the system 1 the transport means 12a, 12b are each embodied as a conduit. Said conduits 10a, 10b each comprise an additional connector 14a, 14b arranged at an outlet end of that conduit 10a, 10b for connecting to a respective infusion element 20a, 20b, in particular to a connector 21a, 21b thereof that fluidly connect to an inlet end of an infusion means 22a, 22b of the infusion element 20a, 20b, herein embodied as a needle, wherein an outlet end of the needle 22a, 22b is configured for being inserted into a body of a person for infusing said substance into said body. The combination of a transport element 10a, 10b and an infusion element 20a, 20b may also be referred to as an infusion set. In order to distinguish between said connectors, the connectors 21a, 21b, 14a, 14b and 11a, 11b may also be referred to as first, second and third connectors, respectively.

In a connected condition in which the connectors 11a, 11b are connected to their respective ports 4a, 4b and the connectors 14a, 14b to connectors 21a, 21b, respectively, a medium through flow connection is provided between the reservoir 100a, 100b and the respective infusion means 22a, 22b, allowing the substance to be fed from the reservoir 100a, 100b via the respective transport element 10a, 10b to the infusion means 22a, 22b and into the body of a person. The device 2 further comprises a controller 5 for controlling the device 2 such that a certain amount of a substance from any of the reservoirs 100a, 100b is infused into the body of the person as described above.

The connector 11, which may be any of the connectors 11a, 11b of FIG. 1, is shown in greater detail in FIGS. 2, 3 and 4. The connector 11 comprises a handle 30 and is provided with aligning and holding means for aligning and holding a reservoir 100, which may be any of the reservoirs 100a, 100b of FIG. 1, connected to the connector 11 in a predetermined orientation with respect to a longitudinal axis of the connector (see FIG. 5D) as well as a predetermined position with respect to the connector 11 for obtaining a substantially fluid-tight connection between the connector 11 and the reservoir 100. Said aligning and holding means in this embodiment comprise a plurality of resilient substantially crescent-shaped members 31 that are attached to and uniformly distributed along the interior of a circumferential wall 32 of the connector 11 with two opposing end zones 33, 34 thereof. In this case, the number of such crescent-shaped members 31 is six, but this number may be different from that, such as larger or smaller than that. A convex side 35 of said substantially crescent-shaped members 31 that extends between the two opposing end zones 33, 34 protrudes inwardly from the inner side of said circumferential wall 32 of the connector 11. The two opposing end zones 33, 34 are arranged at substantially the same position with respect to the longitudinal axis of the connector 11. The resiliency of these substantially crescent-shaped members 31 allows these members 31 to move from a first state for holding the reservoir 100 to a second state when said reservoir 100 is pushed against said members 31 when said reservoir 100 is brought towards said connected condition and/or when said reservoir 100 is disconnected from said connector 11, upon which the resilient members 31 are configured to return to the first state because these are urged to the first state. In the present embodiment, the circumferential wall 32 of the connector 11 is provided with slot-shaped through-holes 36, extending between the opposing end zones 33, 34 of the substantially crescent-shaped members 31. The exterior of the circumferential wall 32 is provided with two connecting means 37 for connecting to the port 4 of the device 2.

In this embodiment, the connector 11 also comprises additional aligning means, embodied in this example as a plurality of elongated ribs 40 that are attached to and uniformly distributed along the inner side of the circumferential wall 31 of the connector 11. These ribs 40 extend substantially parallel to the longitudinal axis of the connector 11 and are configured for being in contact with the reservoir 100 upon insertion into the connector 11, thereby at least pre-aligning the reservoir 100 with respect to the longitudinal axis of the connector 11 in a first part of a connection path of connecting the reservoir 100 to the connector 11. These ribs 40 are arranged with respect to the crescent-shaped members 31 such that in a second, subsequent part of said connection path, said reservoir is aligned by the crescent-shaped members 31 and is subsequently held by the crescent-shaped members 31 at the end of the connection path in the connected condition. In the present embodiment, the ribs 40 extend on both sides of the aligning and holding means 31, such that in addition to said pre-alignment the ribs also provide post-alignment of the reservoir in the connected condition. In the present embodiment, the number of ribs 40 is equal to the number of substantially crescent-shaped members 31. In particular, the ribs 40 and the substantially crescent-shaped members 31 are alternatingly arranged around the circumference of the wall 32, spaced apart from each other.

In particular from FIG. 4 it is clear that as seen in a radially inward direction from the circumferential wall 32, the crescent-shaped members 31 extend further inwards, in particular a center of the convex side 35 thereof, than the ribs 40. This allows the crescent-shaped members 31 to protrude into an area of a neck portion of the reservoir 100 in a fully inserted position of the reservoir into the connector 11 for holding the reservoir.

The connector 11 is further provided with a needle 50, which is in a fluid connection with the inlet end of the transport means 12 and extends through handle 30. Said needle 50 is configured for penetrating a membrane or the like in the cap of the reservoir 100 in the connected condition, wherein said crescent-shaped members 31 are configured for aligning the reservoir 100 in a predetermined orientation with respect to said needle 50 such that the needle 50 is oriented substantially perpendicular to the membrane of said reservoir 100 during penetrating the membrane during connecting the connector 11 to said reservoir, and for holding said reservoir 100 in said predetermined orientation and in such a position that the needle 50 substantially completely penetrates the membrane in the connected condition, in a position or depth with respect to the connector in which a substantially leak-tight fluid connection is obtained between the reservoir 100 and the connector 11. The needle 50 extends from a surface 51 of the connector 11, which surface 51 is opposite to an insertion opening of the connector 11 via which insertion opening the reservoir 100 can be inserted. The needle 50 extends substantially parallel to the longitudinal axis and with a free end thereof in the direction of the crescent-shaped members 31, i.e. in the direction of the insertion opening.

The process of connection of the reservoir 100 to the connector 11 is shown in more detail in FIGS. 5A to 5D. In FIG. 5A, the reservoir 100 is shown separate from the connector 11. It is clear from this figure that reservoir 100 has an outlet end 101 with a neck portion 102, closed off by a head portion 103 comprising a membrane 104 and a side wall 105. In the next step, shown in FIG. 5B, the reservoir 100 is inserted into the connector 11 via the insertion opening thereof, and the reservoir 100 is pre-aligned by a part of the ribs 40 extending before the aligning and holding means 31, as seen in a direction of insertion, as the side wall 105 of the head portion 103 slides along the ribs 40 and is in contact therewith. During this step, the needle 50 does not yet contact the membrane 104. In the next step, shown in FIG. 5C, the insertion proceeds and the side wall 105 of the head portion 103 slides along the substantially crescent-shaped members 31 and is in contact therewith, which crescent-shaped members 31 are, as a consequence thereof, forced from the first state shown in FIGS. 5A and 5B, to a second state and wherein the reservoir 100 is aligned thereby. The second state is not clearly visible in FIG. 5C but the crescent-shaped members 31 are pressed towards the inner side of the circumferential wall 32. The position of the needle 50 with respect to the membrane 104 is such that the needle 50 still does not penetrate the membrane 104 but is about to. As is shown in FIG. 5C, the reservoir 100 is aligned with respect to the needle 50 such that the needle 50 extends substantially orthogonal to the membrane 104 at the location where it is about to pierce the membrane 104. When the reservoir 100 is inserted further into the connector 11, the needle 50 punctures the membrane 104. In a fully inserted position of the reservoir the needle 50 fully extends through the membrane such that a position or depth of the reservoir 100 is achieved where there is a substantially leak-tight fluid connection with the connector 11. Because of the pre-alignment by the ribs 40 and/or the alignment by the substantially crescent-shaped members 31, it is ensured that the membrane is correctly aligned with respect to the longitudinal axis L of the connector 11, i.e. with the surface of the membrane where the needle pierces the membrane 104 substantially perpendicular to this axis. The needle 50 extends substantially parallel to the longitudinal axis L. More in particular, the longitudinal axis of the needle 50 coincides with the longitudinal axis L of the connector 11. As a consequence of the further insertion, the crescent-shaped members 31 return to their first state automatically when the head portion 103 has passed the crescent-shaped members 31 because these are elastically urged to the first state, and then protrude into the area of the neck portion 102 of the reservoir 100 for holding the reservoir 100.

The method of connecting the reservoir 100 to the connector 11, which is shown in FIG. 6, may comprise the step 1001 of providing a transport element, either individually, or as a part of an infusion set according to the invention or a system according to the invention, the step 1002 of providing a reservoir comprising an outlet end, the step 1003 of connecting the outlet end of the reservoir to the connector of the transport element, thereby aligning and holding said reservoir with the aligning and holding means of the connector in a connected condition. The method comprises a step 1004 of inserting the reservoir in said receiving space of the device for selectively supplying at least one substance, which step is preferably carried out after step 1003.

It is noted that the invention is not limited to the shown embodiments but also extends to variants within the scope of the appended claims.

1. A transport element for transporting a substance contained in a reservoir, said transport element being arranged to be part of an infusion set, said transport element comprising a transport means, and a connector arranged at an inlet end of the transport means for connecting to said reservoir such that in a connected condition in which the connector is connected to said reservoir a medium through flow connection is provided between said reservoir and the inlet end of the transport means for receiving said substance from said reservoir, wherein said connector comprises aligning and holding means for aligning and holding the reservoir in a predetermined orientation with respect to and/or position along a longitudinal axis of the connector.

2. The transport element according to claim 1, wherein the connector of the transport element comprises additional aligning means, said additional aligning means extending before and/or after the aligning and holding means as seen in the direction of the longitudinal axis.

3. The transport element according to claim 2, wherein said additional aligning means extending before the aligning and holding means are arranged for pre-aligning the reservoir with respect to the longitudinal axis of the connector in a first part of a connection path of connecting the reservoir to the connector, wherein in a second, subsequent part of said connection path said reservoir is aligned by said aligning and holding means and optionally said additional aligning means, and is subsequently held by the aligning and holding means at the end of the connection path in the connected condition.

4. The transport element according to claim 2, wherein said additional aligning means extending after the aligning and holding means are arranged for maintaining the alignment of said reservoir in said predetermined orientation in the connected condition.

5. The transport element according to claim 2, wherein the aligning and holding means are more flexible than the additional aligning means.

6. The transport element according to claim 2, wherein the additional aligning means comprise at least one elongated rib that protrudes inwardly from the inner side of a circumferential wall of said connector and extends substantially parallel to said longitudinal axis.

7. The transport element according to claim 6, wherein the additional aligning means comprise a plurality of said ribs distributed along the circumference of the circumferential wall, preferably in a uniform distribution.

8. The transport element according to claim 1, wherein the aligning and holding means comprise at least one resilient member, said resilient member having a first state for holding said reservoir and being moveable to a second state when said reservoir is pushed against said resilient member when said reservoir is connected to or disconnected from said connector, and which resilient member is elastically urged to its first state.

9. The transport element according to claim 8, wherein said at least one resilient member comprises at least one substantially crescent-shaped member, wherein the substantially crescent-shaped member is attached to the inner side of a or the circumferential wall of the connector with two opposing end zones thereof and wherein a convex side of said substantially crescent-shaped member that extends between the two opposing end zones protrudes inwardly from the inner side of said circumferential wall of the connector.

10. The transport element according to claim 9, wherein a hole or opening is provided in said circumferential wall in an area between said two opposing end zones of the at least one substantially crescent-shaped member.

11. The transport element according to claim 8, comprising a plurality of said resilient members and wherein the resilient members are distributed along the circumference of a or the circumferential wall.

12. The transport element according to claim 11, wherein the additional aligning means comprise at least one elongated rib that protrudes inwardly from the inner side of a circumferential wall of said connector and extends substantially parallel to said longitudinal axis, and wherein the ribs and resilient members are arranged alternatingly along the circumference of the circumferential wall, preferably in an uniform distribution and/or spaced apart from each other.

13. The transport element according to claim 1, wherein said connector comprises a penetrating means, that extends substantially parallel to the longitudinal axis with a free end thereof in the direction of the aligning and holding means for penetrating a membrane or the like of said reservoir in at least the connected condition, wherein said aligning and holding means and optionally said additional aligning means are configured for aligning the reservoir in a predetermined orientation with respect to said penetrating means such that the penetrating means are oriented substantially perpendicular to the membrane of said reservoir during penetrating the membrane during connecting of the connector to said reservoir, and for holding said reservoir in said predetermined orientation and in such a position that the penetrating means substantially completely penetrates the membrane in the connected condition.

14. The transport element according to claim 1, wherein the aligning and holding means are configured to protrude into an area of and/or grip a neck portion of said reservoir in the connected condition.

15. An infusion set, comprising:
the transport element according to claim 1, further comprising an additional connector arranged at an outlet end of the transport means, and
an infusion element comprising an infusion means for infusing a substance into a body of a person, said infusion means comprising an inlet end for receiving said substance and an outlet end for infusing said substance into said body, wherein said infusion element comprises a connector for connecting to said additional connector of said transport element of the infusion set,
wherein said additional connector of said transport element is connectable to said connector of said infusion element such that in a connected condition in which the additional connector of the transport element is connected to the connector of the infusion element a medium through flow connection is provided between the outlet end of the transport means and the inlet end of the infusion means for feeding the substance to the infusion means.

16. A system for regulating the concentration of glucose in the blood of a person, comprising at least one said infusion set according to claim 15 and a device for selectively supplying at least one substance contained in at least one reservoir, said device comprising:
at least one receiving space, for accommodating a said reservoir;
at least one port, for connecting to a said connector of a said transport element of the infusion set, and
a controller for controlling said device such that a certain amount of a said substance is supplied and thereby infused into the body of the person.

17. A combination of a reservoir, and:
a transport element according to claim 1.

18. A method of at least fluidly connecting a transport element to a reservoir, comprising the steps of:
a) providing a transport element according to claim 1;
b) providing a reservoir comprising an outlet end;
c) connecting the outlet end of the reservoir to the connector of the transport element, thereby aligning and holding said reservoir with the aligning and holding means of the connector in a connected condition.

19. The method according to claim 18, wherein in step c) said reservoir is connected to the connector along a connection path, wherein in a first part of said path the reservoir is pre-aligned by said additional aligning means, wherein in a second, subsequent part of said path the reservoir is aligned by said aligning and holding means and optionally the additional aligning means, and wherein said reservoir is subsequently held by said aligning and holding means in said connected condition and optionally aligned by said additional aligning means.

20. The method according to claim 18 wherein the transport element is part of an infusion set, said infusion set further comprising an additional connector arranged at an outlet end of the transport means, and an infusion element comprising an infusion means for infusing a substance into a body of a person, said infusion means comprising an inlet end for receiving said substance and an outlet end for infusing said substance into said body, wherein said infusion element comprises a connector for connecting to said additional connector of said transport element of the infusion set, wherein said additional connector of said transport element is connectable to said connector of said infusion element such that in a connected condition in which the additional connector of the transport element is connected to the connector of the infusion element a medium through flow connection is provided between the outlet end of the transport means and the inlet end of the infusion means for feeding the substance to the infusion means, wherein said infusion set is part of a system for regulating the concentration of glucose in the blood of a person, said system further comprising a device for selectively supplying at least one substance contained in at least one reservoir, said device comprising at least one receiving space, for accommodating a said reservoir, at least one port, for connecting to a said connector of a said transport element of the infusion set, and a controller for controlling said device such that a certain amount of a said substance is supplied and thereby infused into the body of the person, and wherein the method further comprises step d) of inserting the reservoir in said receiving space of the device.
